(19) United States
(12) Patent Application Publication
(10) Pub. No.: US 2019/0365260 A1
(43) Pub. Date: Dec. 5, 2019

(54) BIOLOGICAL INFORMATION MEASURING APPARATUS, METHOD, AND PROGRAM

(71) Applicants: OMRON CORPORATION, Kyoto (JP); OMRON HEALTHCARE CO., LTD., Kyoto (JP)

(72) Inventors: Tsuyoshi KITAGAWA, Kyoto (JP); Shingo YAMASHITA, Kyoto (JP)

(21) Appl. No.: 16/543,979

(22) Filed: Aug. 19, 2019

Related U.S. Application Data
(63) Continuation of application No. PCT/JP2018/009564, filed on Mar. 12, 2018.

(30) Foreign Application Priority Data
Mar. 15, 2017 (JP) 2017-050594

(51) Int. Cl. A61B 5/022; A61B 5/021; A61B 5/00

(52) U.S. Cl. CPC A61B 5/02233; A61B 2560/0223; A61B 5/6824; A61B 5/02108

(57) ABSTRACT

According to one embodiment, an apparatus includes a detector, a measuring unit, and a calculator. The detector detects a pulse wave in a temporally continuous manner. The measuring unit measures first biological information intermittently. A calculator calibrates the pulse wave based on the first biological information and calculates second biological information from the pulse wave calibrated. The detector, The measuring unit, and The calculator are arranged at a same site.

### CROSS-REFERENCE TO RELATED APPLICATIONS

This application is a Continuation Application of PCT Application No. PCT/JP2018/009564, filed Mar. 12, 2018 and based upon and claiming the benefit of priority from Japanese Patent Application No. 2017-050594, filed Mar. 15, 2017, the entire contents of all of which are incorporated herein by reference.

### FIELD

The present invention relates to a biological information measuring apparatus that continuously measures biological information, a method, and a program.

### BACKGROUND

The development of sensor technology has brought about an environment which allows high-performance sensors to be easily used, and it is getting more and more important in medical treatment to detect biological abnormalities early by utilizing biological information and to put it to use in treatment.

There is known a biological information measuring apparatus capable of measuring biological information such as a pulse and a blood pressure using information detected by a pressure sensor being brought in direct contact with a living body site through which an artery such as a radial artery of the wrist extends (see, for example, Jpn. Pat. Appln. KOKAI Publication No. 2004-113368).

The blood pressure measuring apparatus described in Jpn. Pat. Appln. KOKAI Publication No. 2004-113368 calculates a blood pressure value using a cuff at a site different from a living body site which the pressure sensor is brought in contact with, and generates calibration data from the calculated blood pressure value. Then a pressure pulse wave detected by the pressure sensor is calibrated using this calibration data. In this manner, a blood pressure value is calculated per pulse.

However, the blood pressure measuring apparatus described in Jpn. Pat. Appln. KOKAI Publication No. 2004-113368 requires a plurality of devices, and is too large in size to increase the measurement accuracy. Furthermore, this blood pressure measuring apparatus is premised on operation performed in a limited environment by a specific person. Thus, it is difficult to use the apparatus for daily medical care or at home. In addition, this blood pressure measuring apparatus requires a large volume of tubes and wiring which are burdensome, and is therefore not practical for use on a daily basis or during sleep.

### SUMMARY

According to the first aspect of the present invention, a biological information measuring apparatus includes a detector, a measuring unit, and a calculator all in the same site. The detector detects a pulse wave in a temporally continuous manner. The measuring unit measures first biological information intermittently. The calculator calibrates the pulse wave based on the first biological information and calculates second biological information from the pulse wave calibrated.

According to a second aspect of the present invention, the detector and the measuring unit are included in a same housing.

According to a third aspect of the present invention, the biological information measuring apparatus further includes a connecting unit that physically connects and integrates the detector and the measuring unit.

According to a fourth aspect of the present invention, the detector is disposed on a wrist of a living body, and the measuring unit is disposed closer to an upper arm than the detector.

According to a fifth aspect of the present invention, a length of the detector has a smaller than a length of the measuring unit in an arm-extending direction.

According to a sixth aspect of the present invention, a first portion of the detector, a first portion of the detector differs in height from a third portion of the measuring unit. The first portion is arranged on a palm side, and the third portion is arranged on the palm side.

According to a seventh aspect of the present invention, the third portion is larger in height than the first portion.

According to an eighth aspect of the present invention, a second portion of the detector differs in height from a fourth portion of the measuring unit. The second portion is arranged on a back side of a hand, and the fourth portion is arranged on the back side of the hand.

According to a ninth aspect of the present invention, the detector differs from the measuring unit in terms of height from a surface of an arm, at any position of the arm to which the detector and the measuring unit are arranged.

According to a tenth aspect of the present invention, the measuring unit measures first biological information with higher accuracy than that of second biological information obtained from the detector.

According to an eleventh aspect of the present invention, the detector detects the pulse wave for each pulse, and the first biological information and the second biological information are related to a blood pressure.

According to a twelfth aspect of the present invention, the detector detects a pressure pulse wave as the pulse wave.

According to the first embodiment, the detector that detects a pulse wave in a temporally continuous manner and the measuring unit that measures first biological information intermittently enable the biological information measuring apparatus to be made compact, which allows the biological information measuring apparatus to be easily worn and facilitates measurement, thereby increasing the convenience for a user. The pulse wave is calibrated based on the biological information measured by the measuring unit. This makes it possible to calculate biological information with high accuracy from a pulse wave, so that a user can easily obtain biological information with high accuracy. Furthermore, the measuring unit only measures intermittently. This reduces a time during which a user is interrupted by the measuring unit. In addition, the detector, the measuring unit, and the calculator are provided at the same site (for example, the left wrist or right wrist), so that biological information can be acquired from substantially the same portion.

According to the second aspect of the present invention, the detector and the measuring unit are included in the same housing. This makes the biological information measurement device compact.

According to the third aspect of the present invention, the biological information measuring apparatus further comprises a connecting unit that physically connects and integrates the detector and the measuring unit. This makes the biological information measurement device compact.

According to the fourth aspect of the present invention, the detector is disposed on a wrist of a living body, and the measuring unit is disposed closer to an upper arm than the detector. This ensures the detection of a pulse wave from the wrist.

According to the fifth aspect of the present invention, a length of the detector has a smaller width than that of a length of the measuring unit in an arm-extending direction. This enables the measuring unit to be arranged even closer to the palm. Thus, the pulse wave can be easily detected and the measurement accuracy can be maintained in a good condition.

According to the sixth aspect of the present invention, a first portion of the detector, to be arranged on a palm side, differs in height from a third portion of the measuring unit, to be arranged on the palm side. This makes it easy for a user to determine positions of the detector and the measuring unit visually and haptically, thereby facilitating the positioning of the detector and the measuring unit. Therefore, it becomes easy to arrange the sensor at a specific position. As a result, biological information can be easily measured, and the measurement accuracy can be maintained in a good state.

According to the seventh aspect of the present invention, the third portion is larger in height than the first portion. This makes it easy to discriminate between the detector and the measuring unit and to arrange the sensor at a specific position.

According to the eighth aspect of the present invention, a second portion of the detector, to be arranged on a back side of a hand, differs in height from a fourth portion of the measuring unit, to be arranged on the back side of the hand. This makes it easy to discriminate between the detector and the measuring unit and to arrange the sensor at a specific position.

According to the ninth aspect of the present invention, the detector differs from the measuring unit in terms of height from a surface of an arm, at any position of the arm to which the detector and the measuring unit are arranged. This makes it easy for a user to determine a position of the detector visually and haptically, thereby facilitating the positioning of the sensor.

According to the tenth aspect of the present invention, the measuring unit measures the second biological information with higher accuracy than that of the first biological information obtained from the detector. This ensures the accuracy of biological information obtained based on a pulse wave from the detector. Therefore, it becomes possible to calculate biological information with high accuracy in a temporally continuous manner.

According to the eleventh aspect of the present invention, the detector detects the pulse wave for each pulse, the pulse being generated along with the heartbeat, and the first biological information and the second biological information are related to a blood pressure. This enables the biological information measuring apparatus to measure a blood pressure for each pulse wave per pulse in a temporally continuous manner.

According to the twelfth aspect of the present invention, the detector detects a pressure pulse wave as the pulse wave. This enables the detection of blood pressure per pulse based on a pressure pulse wave in a temporally continuous manner.

That is, according to each aspect of the present invention, it is possible to provide a biological information measuring apparatus capable of acquiring accurate information while calibrating biological information in a temporally continuous manner with the apparatus being worn constantly, a method therefor, and a program therefor.

### BRIEF DESCRIPTION OF THE DRAWINGS

FIG. 1 is a block diagram showing a blood pressure measuring apparatus according to an embodiment.

FIG. 2 is a view showing an example in which the blood pressure measuring apparatus of FIG. 1 is worn on a wrist.

FIG. 3 is a view showing another example in which the blood pressure measuring apparatus of FIG. 1 is worn on the wrist.

FIG. 4 is a view showing the time course of a cuff pressure and a pulse wave signal by the oscillometric technique.

FIG. 5 is a view showing a time variation of a pulse pressure for each of pulses and a pulse wave of one of the pulses.

FIG. 6 is a flowchart showing a calibration method.

FIG. 7A is a cross-sectional view of a state in which a pulse wave detector of FIG. 1 is worn on an arm.

FIG. 7B is a cross-sectional view of a state in which a blood pressure measuring device of FIG. 1 is worn on an arm.

FIG. 8 is a view showing that the pulse wave detector is higher than the blood pressure measuring device in the state shown in FIG. 2.

### DETAILED DESCRIPTION

Hereinafter, a biological information measuring apparatus, a method therefor, and a program therefor according to embodiments of the present invention will be described with reference to the drawings. In the following embodiments, the portions given the same numbers operate similarly, and any overlapping description will be omitted.

The present embodiments have been made in view of the above circumstances, and aim to provide a biological information measuring apparatus capable of acquiring accurate information while calibrating biological information in a temporally continuous manner with the apparatus being worn constantly, a method therefor, and a program therefor.

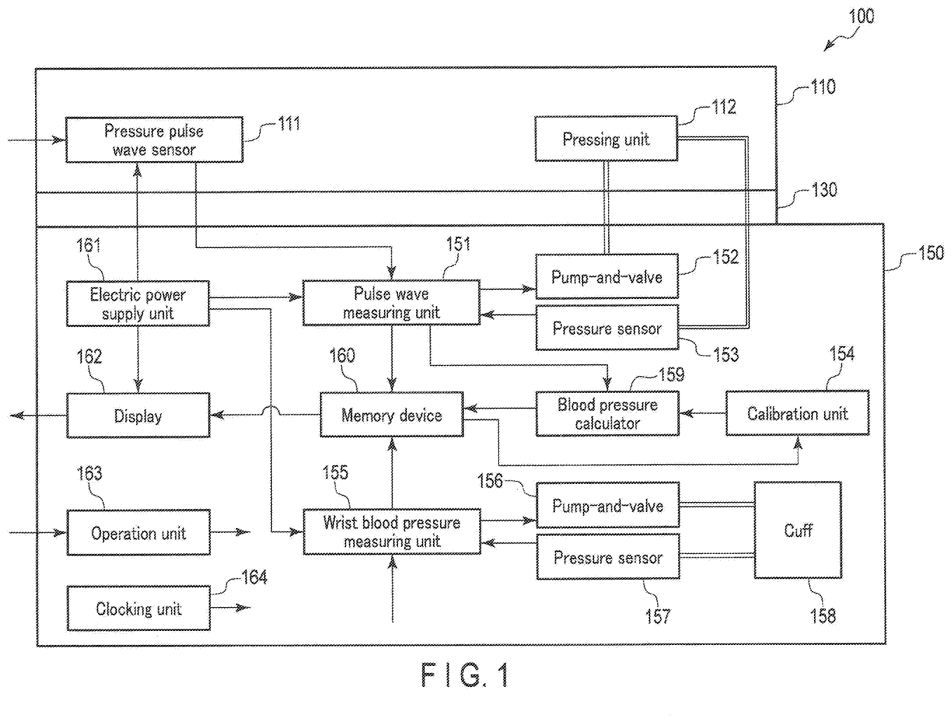

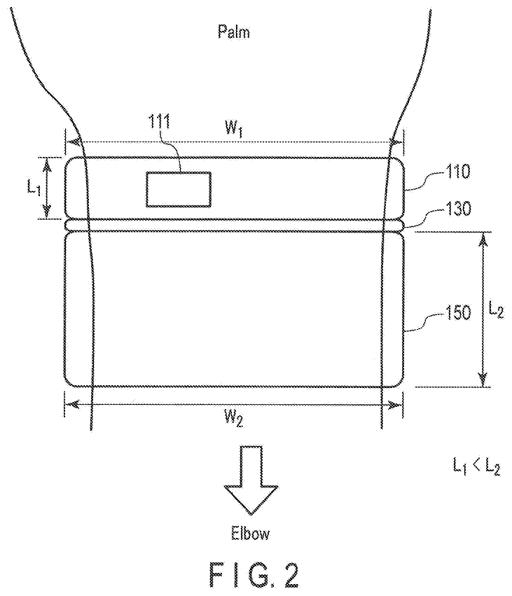

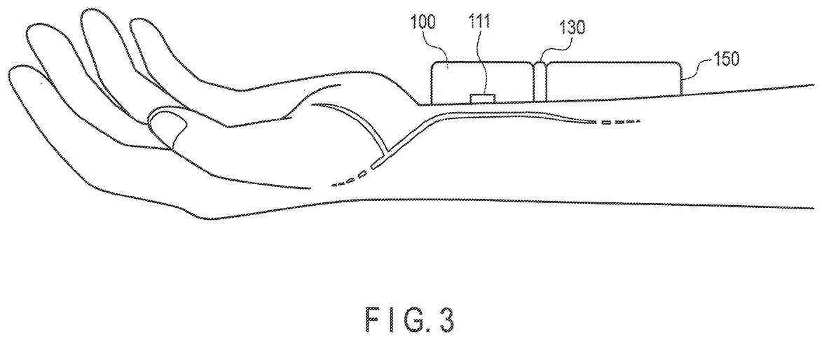

A blood pressure measuring apparatus 100 according to the present embodiment will be described with reference to FIGS. 1, 2, and 3. FIG. 1 is a functional block diagram of the blood pressure measuring apparatus 100, and illustrates details of a pulse wave detector 110 and a blood pressure measuring device 150. FIG. 2 shows an example in which the blood pressure measuring apparatus 100 is worn on the wrist, and is a schematic perspective view from above the palm. A pressure pulse wave sensor 111 is arranged on a side closer to the wrist, of the pulse wave detector 110. FIG. 3 is an image diagram of the blood pressure measuring apparatus 100 being worn, and is a schematic perspective view in which the palm is viewed from the side (the direction in which the fingers line up with the hand being opened). FIG. 3 shows an example in which the pressure pulse wave sensor 111 is arranged to be orthogonal to the radial artery. In FIG. 3, the blood pressure measuring apparatus 100 appears to be simply placed on the palm side of the arm. In reality, however, the blood pressure measuring apparatus 100 is wound around the arm.

The blood pressure measuring apparatus 100 includes the pulse wave detector 110, a connecting unit 130, and the blood pressure measuring device 150. The pulse wave detector 110 includes the pressure pulse wave sensor 111 and a pressing unit 112. The blood pressure measuring device 150 includes a pulse wave measuring unit 151, a pump-and-valve 152, a pressure sensor 153, a calibration unit 154, a wrist blood pressure measuring device 155, a pump-and-valve 156, a pressure sensor 157, a cuff 158, a blood pressure calculator 159, a memory device 160, an electric power source unit 161, a display 162, an operation unit 163, and a clocking unit 164. Furthermore, the pulse wave detector 110 and the blood pressure measuring device 150 may be arranged in a manner such that they are included in the same housing. The connecting unit 130 may not be installed.

The blood pressure measuring apparatus 100 forms an annular shape and measures the blood pressure by being wound like a bracelet around the wrist, etc. As shown in FIGS. 2 and 3, the pulse wave detector 110 is arranged on a side of the wrist which is closer to the palm than the blood pressure measuring device 150. In other words, the pulse wave detector 110 is arranged at a position farther from the elbow than the blood pressure measuring device 150. In the present embodiment, the pulse wave detector 110 is arranged in a manner such that the pressure pulse wave sensor 111 is located on the radial artery. This arrangement brings the blood pressure measuring device 150 closer to the elbow than the pulse wave detector 110. The connecting unit 130 physically connects the pulse wave detector 110 and the blood pressure measuring device 150, and is made of, for example, a shock absorbing material, in order to prevent interference with their measurements.

The pulse wave detector 110 has length L₁ in the arm-extending direction, while the blood pressure measuring device 150 has length L₂ in the extending direction. The length L₁ is set smaller than length L₂. The Length L₁ of the pulse wave detector 110 in the arm-extending direction is set to 40 mm or less, ideally, 15 to 25 mm. The pulse wave detector 110 has length W₁ in the direction perpendicular to the arm-extending direction, while the blood pressure measuring device 150 has length W₂ in the direction perpendicular to the extending direction. The length W₁ is set between 4 and 5 cm, while the length W₂ is set between 6 and 7 cm. Furthermore, the lengths W₁ and W₂ have a relationship of 0 (or 0.5) cm<W₂−W₁<2 cm. According to this relationship, the length W₂ is set not to be too long, thereby making it difficult to cause interference with surroundings. By the pulse wave detector 110 being fitted in a width to this extent, the blood pressure measuring device 150 is arranged even closer to the palm, so that the pulse wave can be easily detected and the measurement accuracy can be maintained.

The pressure pulse wave sensor 111 detects the pressure pulse wave in a temporally continuous manner. For example, the pressure pulse wave sensor 111 detects a pressure pulse wave for each pulse. The pressure pulse wave sensor 111 is arranged closer to the palm, as shown in FIG. 2, and is normally arranged in parallel to the arm-extending direction as shown in FIG. 3. With the pressure pulse wave sensor 111, time-series data of a blood pressure value (blood pressure waveform) that varies in conjunction with a heart rate can be obtained.

By acquiring, from the clocking unit 164, a time when the pulse wave measuring unit 151 receives a pressure pulse wave from the pressure pulse wave sensor 111, it is possible to estimate a time when the pressure pulse wave sensor 111 detects the pressure pulse wave.

The pressing unit 112 is an air bag and can increase the sensor sensitivity by pressing the sensor portion of the pressure pulse wave sensor 111 against the wrist.

The pulse wave measuring unit 151 receives, from the pressure pulse wave sensor 111, pressure pulse wave data along with a time, and transmits this data to the memory device 160 and the blood pressure calculator 159. The pulse wave measuring unit 151 pressurizes or depressurizes the pressing unit 112 by controlling the pump-and-valve 152 and the pressure sensor 153, and adjusts the pressure pulse wave sensor 111 in a manner to press the radial artery of the wrist.

The pump-and-valve 152 pressurizes or depressurizes the pressing unit 112 according to an instruction from the pulse wave measuring unit 151. The pressure sensor 153 monitors the pressure of the pressing unit 112 and notifies the pulse wave measuring unit 151 of a pressure value of the pressing unit 112.

The wrist blood pressure measuring device 155 measures the blood pressure as biological information with higher accuracy than the pressure pulse wave sensor 111. For example, the wrist blood pressure measuring device 155 measures the blood pressure intermittently, not in a temporally continuous manner, and transmits the measured value to the calibration unit 154. The wrist blood pressure measuring device 155 measures the blood pressure using, for example, the oscillometric technique. The wrist blood pressure measuring device 155 pressurizes or depressurizes the cuff 158 by controlling the pump-and-valve 156 and the pressure sensor 157, thereby measuring the blood pressure. The wrist blood pressure measuring device 155 transmits to the memory device 160, the systolic blood pressure together with a time when this systolic blood pressure is measured, and the diastolic blood pressure together with a time when this diastolic blood pressure is measured. The systolic blood pressure is also referred to as SBP, while the diastolic blood pressure is also referred to as DBP.

The memory device 160 sequentially acquires from the pulse wave measuring unit 151, the pressure pulse wave data together with a detection time, and stores them. The memory device 160 also acquires from the wrist blood pressure measuring device 155, an SBP measurement time acquired when this measuring unit operates, together with the SBP, as well as a DBP measurement time together with the DBP.

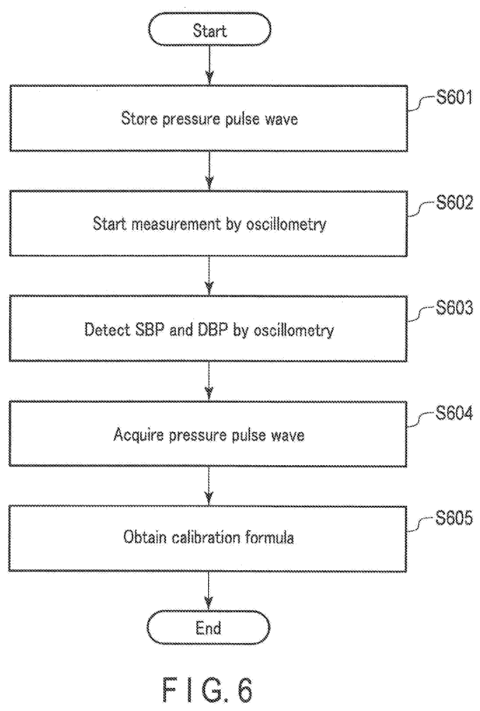

The calibration unit 154 acquires, from the memory device 160, the SBP and DBP measured by the wrist blood pressure measuring device 155 together with the measurement times, and the pressure pulse wave data measured by the pulse wave measuring unit 151 together with the measurement time. The calibration unit 154 calibrates the pressure pulse wave from the pulse wave measuring unit 151 based on the blood pressure value from the wrist blood pressure measuring device 155. Although there are several possible calibration methods performed by the calibration unit 154, one calibration method will be described in detail later with reference to FIG. 6.

The blood pressure calculator 159 receives the calibration method from the calibration unit 154, calibrates the pressure pulse wave data from the pulse wave measuring unit 151, and causes the memory device 160 to store the blood pressure data obtained from the calibrated pressure pulse wave data, together with the measurement time.

The electric power source unit 161 supplies power to each of the pulse wave detector 110 and the blood pressure measuring device 150.

The display 162 displays a blood pressure measurement result, and displays various types of information to a user. For example, the display 162 receives data from the memory device 160 and displays the contents of the data. For example, the display 162 displays pressure pulse wave data together with measurement time.

The operation unit 163 receives an operation from a user. The operation unit 163 includes, for example, an operation button for causing the wrist blood pressure measuring device 155 to start measurement, and an operation button for performing calibration.

The clocking unit 164 generates and supplies a time to a unit which requires it. For example, the memory device 160 records a time as well as data to be stored.

To implement the pulse wave measuring unit 151, the calibration unit 154, the blood pressure calculator 159, and the wrist blood pressure measuring device 155 described herein, each of them includes a secondary memory device to store therein a program for executing the operations described above, and causes a central processing unit (CPU) to execute computation by reading the stored program. The secondary memory device is, for example, a hard disk. However, any memory device can be used, and may be, for example, a semiconductor memory, a magnetic memory device, an optical memory device, an optical magnetic disk, and a memory device to which the phase change recording technology is applied.

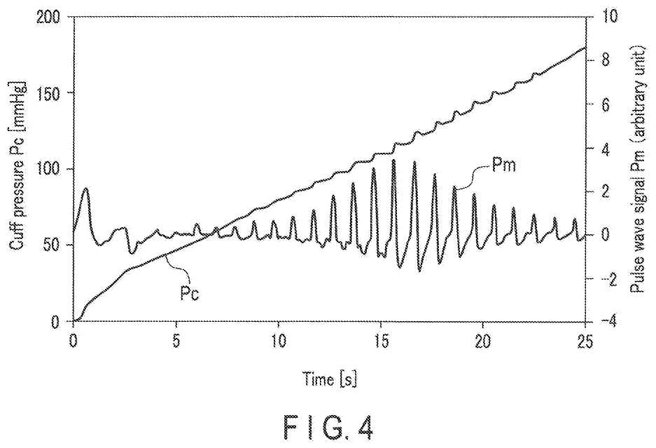

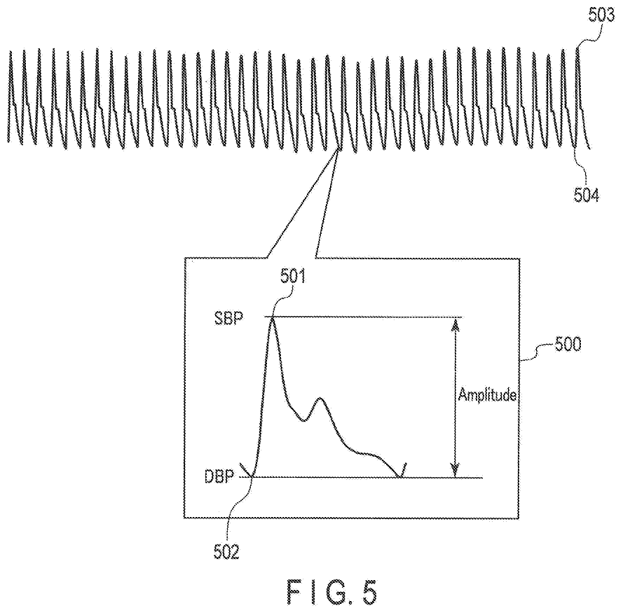

Described next with reference to FIGS. 4 and 5 are contents performed by the pulse wave measuring unit 151 and the wrist blood pressure measuring device 155 before the calibration unit 154 performs calibration. FIG. 4 shows the time variation of the cuff pressure and the time variation of the magnitude of the pulse wave signal in the blood pressure measurement by the oscillometric technique. FIG. 4 shows the time variation of the cuff pressure and the time variation of the pulse wave signal, and illustrates that the cuff pressure increases with time while the magnitude of the pulse wave signal gradually increases to the maximum value with the increase of the cuff pressure and then decreases gradually. FIG. 5 shows time-series data of the pulse pressure when the pulse pressure is measured for each pulse. Furthermore, FIG. 5 shows the waveform of one of the pressure pulses.

First, the operation at the time when the wrist blood pressure measuring device 155 measures the blood pressure by the oscillometric technique will be briefly described with reference to FIG. 4. A blood pressure value may be calculated not only in a pressurizing process but also in a depressurizing process. However, only the pressurizing process is described herein.

When a user instructs blood pressure measurement by the oscillometric technique using the operation unit 163 provided in the blood pressure measuring device 150, the wrist blood pressure measuring device 155 starts operating and initializes the processing memory area. The wrist blood pressure measuring device 155 turns off the pump of the pump-and-valve 156 to open the valve, thereby exhausting the air in the cuff 158. Subsequently, the wrist blood pressure measuring device 155 performs control to set a present output value of the pressure sensor 157 as a value corresponding to the atmospheric pressure (0 mmHg adjustment).

The wrist blood pressure measuring device 155 then operates as a pressure control unit and performs control by closing the valve of the pump-and-valve 156 and then driving the pump to deliver air to the cuff 158. This inflates the cuff 158 and gradually increases the cuff pressure (Pc in FIG. 4) to apply the pressure. In this pressurizing process, the wrist blood pressure measuring device 155 monitors the cuff pressure Pc via the pressure sensor 157 in order to calculate a blood pressure value, and acquires as a pulse wave signal Pm as shown in FIG. 4, a fluctuation component of the arterial volume, generated in the radial artery in the wrist as a site to be measured.

Next, based on the pulse wave signal Pm acquired at this point, the wrist blood pressure measuring device 155 tries to calculate the blood pressure values (SBP and DBP) by applying a known algorithm by the oscillometric technique. If the blood pressure value cannot be calculated at this point because of insufficient data, as long as the cuff pressure Pc has not yet reached the upper limit pressure (predetermined to be, for example, 300 mmHg, for safety), the same pressurizing processing as above is repeated.

When a blood pressure value is calculated in this manner, the wrist blood pressure measuring device 155 performs control to exhaust the air in the cuff 158 by stopping the pump of the pump-and-valve 156 and opening the valve. Finally, the wrist blood pressure measuring device 155 transmits the measurement result of the blood pressure value to the calibration unit.

Next, the operation of the pulse wave measuring unit 151 to measure the pulse wave for each pulse will be described with reference to FIG. 5. The pulse wave measuring unit 151 measures the pulse wave by, for example, the tonometry method.

The pulse wave measuring unit 151 controls the pump-and-valve 152 and the pressure sensor 153 to increase the internal pressure of the pressing unit 112 to the optimum pressing force determined in advance for the pressure pulse wave sensor 111 to realize the optimum measurement, and maintains this optimum pressing force. Next, when a pressure pulse wave is detected by the pressure pulse wave sensor 111, the pulse wave measuring unit 151 acquires this pressure pulse wave.

The pressure pulse wave is detected for each pulse as a waveform shown in FIG. 5, and respective pressure pulse waves are detected continuously. In FIG. 5, the pressure pulse wave 500 is a pressure pulse wave of one pulse. The pressure value of 501 corresponds to SBP, and the pressure value of 502 corresponds to DBP. As shown in the pressure pulse wave time series of FIG. 5, normally, SBP 503 and DBP 504 fluctuate for each pressure pulse wave.

Next, the operation of the calibration unit 154 will be described with reference to FIG. 6.

The calibration unit 154 calibrates the pressure pulse wave detected by the pulse wave measuring unit 151, using a blood pressure value measured by the wrist blood pressure measuring device 155. That is, the calibration unit 154 determines blood pressure values of the maximum value 501 and the minimum value 502 of the pressure pulse wave detected by the pulse wave measuring unit 151.

(Calibration Method)

The pulse wave measuring unit 151 starts recording the pressure pulse wave data of the pressure pulse wave, and sequentially stores the pressure pulse wave data in the memory device 160 (step S601). Thereafter, for example, a user activates the wrist blood pressure measuring device 155 using the operation unit 163 to start measurement by the oscillometric technique (step S602). Based on the pulse wave signal Pm, the wrist blood pressure measuring device 155 records SBP data and DBP data obtained by detecting SBP and DBP by the oscillometric technique, respectively, and stores these SBP data and DBP data in the memory device 160 (step S603).

The calibration unit 154 acquires pressure pulse waves corresponding to the SBP data and the DBP data from the pressure pulse wave data (step S604). The calibration unit 154 obtains a calibration formula based on the maximum value 501 of the pressure pulse wave corresponding to SBP and the minimum value 502 of the pressure pulse wave corresponding to DBP (step S605).

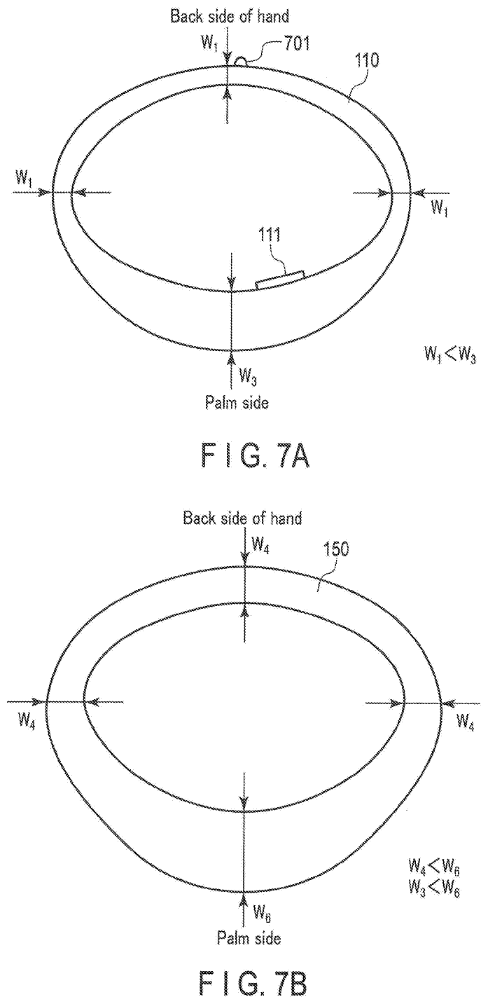

Next, the shape of the blood pressure measuring apparatus 100 according to the present embodiment will be described with reference to FIGS. 7A and 7B. Each of FIGS. 7A and 7B is a cross-sectional view perpendicular to the arm-extending direction when the pulse wave detector 110 and the blood pressure measuring device 150 are worn on the wrist, and shows the cross section of each of the pulse wave detector 110 and the blood pressure measuring device 150 as seen in the cross-section of the arm.

As shown in FIG. 7A, the pulse wave detector 110 of the blood pressure measuring apparatus 100 differs in shape between a part arranged on the back side of the hand and a part arranged on the palm side. For example, as shown in FIG. 7A, the pulse wave detector 110 is characterized in that the height (thickness) from the surface of the arm on the back side of the hand is small, and the thickness on the palm side is large. More specifically, the pulse wave detector 110 has uniform thickness W1 on the back side of the hand, a thickness that increases from a transition position from the back side of the hand to the palm side, and a thickness as expressed by W₃ (W₁<W₃) around the center of the palm.

In a similar manner to the pulse wave detector 110, the blood pressure measuring device 150 of the blood pressure measuring apparatus 100 differs in shape between a part arranged on the back side of the hand and a part arranged on the palm side, and has a similar shape to that of the pulse wave detector 110, as shown in FIG. 7B. That is, for example, as shown in FIG. 7B, the blood pressure measuring device 150 is designed to have a small thickness on the back side of the hand and a large thickness on the palm side. More specifically, the blood pressure measuring device 150 has uniform thickness W₄ on the back side of the hand, a thickness that increases from a transition position from the back side of the hand to the palm side, and a thickness expressed by W₆ (W₄<W₆) around the center of the palm. However, the pulse wave detector 110 and the blood pressure measuring device 150 are not equal in shape, and the blood pressure measuring device 150 is larger in height (thickness) than the pulse wave detector 110. For example, W₃<W₆.

The above structural characteristics of the pulse wave detector 110 and the blood pressure measuring device 150 make it easy for a user to visually recognize a position of the pressure pulse wave sensor 111 of the pulse wave detector 110, which facilitates the positioning of the pressure pulse wave sensor 111. Thus, a blood pressure value with higher accuracy can be obtained. In addition, even a visually handicapped user can recognize a position of the pulse wave detector 110 through the tactile sense of the hand. Thus, good blood pressure measurement can be performed regardless of the state of user's vision.

As shown in FIG. 7A, only the pulse wave detector 110 may be provided with a projection 701. This projection 701 facilitates discrimination between the pulse wave detector 110 and the blood pressure measuring device 150. In addition, by installing the projection 701 at the top of the back side of the hand at its uppermost part, the blood pressure measuring apparatus 100 can be easily positioned with respect to the wrist in the rotation direction (perpendicular to the longitudinal direction of the arm, as the azimuth direction of the arm ring). As a result, the pressure pulse wave sensor 111 can be easily aligned to the position of the radial artery. A similar effect can be obtained by providing a recess instead of the projection 701 at a similar position. Alternatively, a similar projection 701 (or a recess) may be provided on the palm side instead of the back side of the hand, and a similar effect can be obtained in this manner.

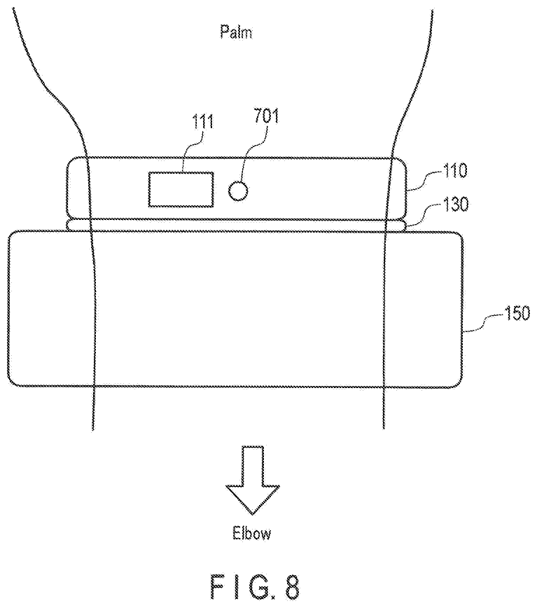

Next, the shape of the blood pressure measuring apparatus 100 according to the present embodiment will be described with reference to FIG. 8. FIG. 8 shows an example in which the blood pressure measuring apparatus 100 is worn on the wrist, and is a schematic perspective view seen from above the palm.

The blood pressure measuring apparatus 100 of the present embodiment is characterized in that the blood pressure measuring device 150 is larger than the pulse wave detector 110 in terms of the height (thickness) from the surface of the arm. In this example, the entire blood pressure measuring device 150 is larger in thickness than the pulse wave detector 110. In such a case, a position of the pulse wave detector 110 becomes visually clear to a user, thereby facilitating the positioning of the pressure pulse wave sensor 111. Thus, a blood pressure value with higher accuracy can be obtained. Since FIG. 8 is a perspective view, it illustrates the projection 701 on the back side of the hand. The blood pressure measuring device 150 becomes less affected by the pulse wave detector 110, and accurate calibration can be expected. In addition, the cuff of the blood pressure measuring device 150 is expanded to have fewer contacts with the pulse wave detector 110, so that displacement of the pulse wave detector 110 is less likely to occur, and detection of the sensor becomes accurate.

In the embodiment described above, the pressure pulse wave sensor 111 detects, for example, a pressure pulse wave of the radial artery extending through a site to be measured (for example, the left wrist) (the tonometry method). However, the present invention is not limited to this. The pressure pulse wave sensor 111 may detect a pulse wave of the radial artery extending through a site to be measured (for example, the left wrist) as a change in impedance (the impedance method). The pressure pulse wave sensor 111 may include a light emitting element that emits light toward the artery extending through a corresponding portion of a site to be measured, and a light receiving element that receives reflected light (or transmitted light) of the emitted light so that a pulse wave of the artery is detected as a change in volume (the photoelectric method). The pressure pulse wave sensor 111 may include a piezoelectric sensor brought in contact with a site to be measured so that strain due to the pressure of the artery extending through a corresponding portion of the site to be measured is detected as a change in electric resistance (the piezoelectric method). The pressure pulse wave sensor 111 may include a transmitting element that transmits a radio wave (transmission wave) toward the artery extending through a corresponding portion of a site to be measured, and a receiving element that receives a reflected wave of the transmitted radio wave so that a change in a distance between the artery and the sensor, obtained from a pulse wave of the artery is detected as a phase shift between the transmitted wave and the reflected wave (the radio wave irradiation method). Any method can be employed other than the above methods as long as such method provides observation of a physical quantity used to calculate the blood pressure.

In the embodiment described above, the blood pressure measuring apparatus 100 is assumed to be attached to the left wrist as a site to be measured. However, the present invention is not limited to this. For example, the blood pressure measuring apparatus 100 may be attached to the right wrist. A site to be measured may be the upper limb such as the upper arm other than the wrist, or the lower limb such as the ankle or thigh as long as such site has an artery extending therethrough.

According to the embodiment described above, the pulse wave detector 110 that detects a pulse wave in a temporally continuous manner and the blood pressure measuring device 150 that measures biological information (first biological information) intermittently are physically connected to be integrated with each other. This makes the biological information measuring apparatus compact and thus facilitates the measurement, thereby increasing the convenience for a user. A pulse wave is calibrated based on the biological information. Biological information (second biological information) is calculated from the pulse wave. The pulse wave is calibrated based on the biological information measured by the blood pressure measuring device 150. This makes it possible to calculate biological information with high accuracy from a pulse wave, so that a user can easily obtain biological information with high accuracy. The blood pressure measuring device 150 only measures intermittently. This reduces a time during which a user is interrupted by the measuring unit.

The pulse wave detector 110 is arranged on the wrist of a living body and the blood pressure measuring device 150 is arranged closer to the upper arm than the pulse wave detector 110. This ensures the detection of a pulse wave from the wrist. The length of the pulse wave detector 110 has a smaller width than the length of the blood pressure measuring device 150 in the arm-extending direction. This enables the blood pressure measuring device 150 to be arranged even closer to the palm. Thus, the pulse wave can be easily detected and the measurement accuracy can be maintained in a good condition. The pulse wave detector 110 differs in height between a first portion to be arranged on the palm side and a second portion to be arranged on the back side of the hand. The blood pressure measuring device 150 differs in height between a third portion to be arranged on the palm side and a fourth portion to be arranged on the back side of the hand. The first portion and the third portion differ in height. The second portion and the fourth portion differ in height. This makes it easy for a user to determine positions of the pulse wave detector 110 and the blood pressure measuring device 150 visually and haptically, thereby facilitating the positioning between the pulse wave detector 110 and the blood pressure measuring device 150.

The pulse wave detector 110 differs from the blood pressure measuring device 150 in terms of height from the surface of the arm at any position of the arm to which they are disposed. This makes it easy for a user to determine a position of the pulse wave detector 110 visually and haptically, thereby facilitating the positioning of the pressure pulse wave sensor 111. Biological information is measured with higher accuracy than that of biological information obtained from the pulse wave detector 110, and biological information of high accuracy is obtained from the blood pressure measuring device 150 and is calibrated. This ensures the accuracy of biological information obtained based on a pulse wave from the pulse wave detector 110. Therefore, it becomes possible to calculate biological information with high accuracy in a temporally continuous manner. The pulse wave detector 110 detects a pulse wave for each pulse, and the biological information is related to the blood pressure. This enables the biological information measuring apparatus to measure the blood pressure for each pulse wave per pulse in a temporally continuous manner. With the biological information measuring apparatus being constantly worn, accurate information can be acquired while calibrating biological information in a temporally continuous manner.

The apparatus of the present invention can also be realized by a computer and a program, and the program can be recorded on a recording medium or provided through a network.

Furthermore, each of the above-described apparatus and their apparatus portions can be implemented either as a hardware configuration or as a combined configuration of hardware resources and software. Used as software of a combined configuration is a program installed in advance from a network or computer-readable recording medium into a computer and is executed by a processor of the computer to cause the computer to realize the functions of the apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

A part or all of the above-mentioned embodiments may also be described as in the following additional notes, without limitation thereto.

### (Additional Note 1)

A biological information measuring device comprising: a hardware processor; and a memory,

wherein the hardware processor is configured to:

detect a pulse wave in a temporally continuous manner;

measure first biological information intermittently; and

calibrate the pulse wave based on the first biological information, and calculate second biological information from the pulse wave, and

wherein the memory includes

a memory device that stores the second biological information.

### (Additional Note 2)

A method of measuring biological information, comprising: detecting a pulse wave in a temporally continuous manner using at least one hardware processor;

measuring first biological information intermittently using at least one hardware processor; and

calibrating the pulse wave based on the first biological information using at least one hardware processor and calculating second biological information from the pulse wave.

1. A biological information measuring apparatus comprising:
a detector configured to detect a pulse wave in a temporally continuous manner;
a measuring unit configured to measure first biological information intermittently; and
a calculator configured to calibrate the pulse wave based on the first biological information and calculate second biological information from the pulse wave calibrated,
wherein the detector, the measuring unit, and the calculator are arranged at a same site.

2. The apparatus according to claim 1, wherein the detector and the measuring unit are configured to be included in a same housing.

3. The apparatus according to claim 1, further comprising a connecting unit configured to physically connect and integrate the detector and the measuring unit.

4. The apparatus according to claim 1, wherein the detector is configured to be disposed on a wrist of a living body, and the measuring unit is configured to be disposed closer to an upper arm than the detector.

5. The apparatus according to claim 4, wherein a length of the detector has a smaller than a length of the measuring unit in an arm-extending direction.

6. The apparatus according to claim 1, wherein a first portion of the detector differs in height from a third portion of the measuring unit, the first portion being arranged on a palm side, the third portion being arranged on the palm side.

7. The apparatus according to claim 6, wherein the third portion is larger in height than the first portion.

8. The apparatus according to claim 1, wherein a second portion of the detector differs in height from a fourth portion of the measuring unit, the second portion being arranged on a back side of a hand, the fourth portion being arranged on the back side of the hand.

9. The apparatus according to claim 1, wherein the detector is configured to differ from the measuring unit in terms of height from a surface of an arm, at any position of the arm on which the detector and the measuring unit are arranged.

10. The apparatus according to claim 1, wherein the measuring unit is configured to measure first biological information with higher accuracy than that of second biological information obtained from the detector.

11. The apparatus according to claim 1, wherein the detector is configured to detect the pulse wave for each pulse, and the first biological information and the second biological information are related to a blood pressure.

12. The apparatus according to claim 1, wherein the detector is configured to detect a pressure pulse wave as the pulse wave.

13. The apparatus according to claim 1, wherein the measuring unit is configured to measure the first biological information intermittently based on a time variation of a cuff pressure and a time variation of a pulse wave signal.

14. A method of a biological information measuring apparatus to measure biological information, the apparatus physically connecting and integrating a detector configured to detect a pulse wave, a measuring unit configured to measure first biological information, and a calculator that calculates second biological information from the pulse wave, the method comprising:
detecting the pulse wave in a temporally continuous manner;
measuring the first biological information intermittently;
calibrating the pulse wave based on the first biological information; and
calculating second biological information from the pulse wave calibrated, the detector, the measuring unit, and the calculator being arranged at a same site.

15. The apparatus according to claim 14, wherein the measuring the first biological information measures the first biological information intermittently based on a time variation of a cuff pressure and a time variation of a pulse wave signal.

16. A non-transitory computer readable medium storing a computer program which is executed by a computer to provide the steps of, the computer physically connecting and integrating a detector configured to that detects a pulse wave, and a measuring unit configured to that measures first biological information, and a calculator that calculates second biological information from the pulse wave:
detecting the pulse wave in a temporally continuous manner;
measuring the first biological information intermittently; and
calibrating the pulse wave based on the first biological information; and
calculating second biological information from the pulse wave calibrated, the detector, the measuring unit, and the calculator being arranged at a same site.
